(19) United States
(12) Patent Application Publication
(10) Pub. No.: US 2025/0261877 A1
(43) Pub. Date: Aug. 21, 2025

(54) SYSTEMS AND METHODS FOR DETECTING A POSITION OF A SUBJECT

(71) Applicant: MERCURY ALERT, INC., Arlington, VA (US)

(72) Inventor: Brandon Julius PECK, Arlington, VA (US)

(73) Assignee: MERCURY ALERT, INC., Arlington, VA (US)

(21) Appl. No.: 18/993,962

(22) PCT Filed: Jul. 13, 2023
(86) PCT No.: PCT/US2023/027631

§ 371 Date: Jan. 13, 2025

Related U.S. Application Data
(60) Provisional application No. 63/368,317, filed on Jul. 13, 2022.

(51) Int. Cl. A61B 5/11; G06V 20/52; G08B 21/04

(52) U.S. Cl. CPC A61B 5/1117; A61B 5/1128; G06V 20/52; G08B 21/043; G08B 21/0476; G06T 2207/20081; G06T 2207/20084; G06T 2207/20132

(57) ABSTRACT

A system for detecting changes in the position of a subject is provided. An imaging device captures one or more images in a room where a subject may be located and provides the one or more images to an artificial neural network for processing. The ANN processes the images to determine a current state of a subject in the room. If the determined current state of the subject is associated with one or more alerts, the system delivers one or more alerts, which may be audible, visual, and/or haptic.

### RELATED APPLICATION

The present application claims priority to U.S. provisional patent application No. 63/368,317 filed Jul. 13, 2022, which is incorporated herein by reference in its entirety.

### BACKGROUND

### Field of the Invention

The present disclosure is generally directed toward machine learning and computer vision and more specifically related to systems and methods for monitoring a subject to detect changes in a position of the subject.

### Related Art

Care facilities, other facilities, and private residences where individuals are housed have a need to monitor the well being of their occupants. In some circumstances, occupants of such facilities and homes may fall or find themselves in situations without anyone nearby being aware or able to assist. Existing technologies designed to address these issues generally require wearable devices with accelerometers and buttons. When the accelerometers activate or when the buttons are pressed, they trigger alert messages that are designed to elicit prompt responses. However, these solutions necessitate that the occupants consistently wear one or more devices and that an occupant has the capacity to engage with a device by pressing a button. This interactivity requirement for emergency alerting presents significant issues. Accordingly, what is needed is a system and method that overcomes these significant problems found in the conventional systems as described above.

### SUMMARY

In one aspect, the field of view of a camera sensor is trained on a at least a portion of a room where a subject may be located. The camera is configured to periodically and automatically capture images of the field of view of the camera sensor within the room. The captured images may be color, infrared, black and white, or some other type of image corresponding to the ambient environment of the room and the capabilities of the camera sensor. The captured images are processed by a trained machine learning model, such as an artificial neural network (“ANN”), e.g., a convolutional neural network, to determine if a subject is present by being at least partially within the field of view of the sensor, and to determine a position of the subject when the subject is present. The input to the ANN can be multiple images taken over time or a single image and the ANN architecture is configured to receive and process such input.

The possible positions of the subject can include at least sitting, standing, in-bed, and fallen. Other positions may also be defined as desired to convey the orientation and safety of the subject. The possible positions of the subject are referred to herein as “states” and additional states may include, away (i.e., not present), with caregiver present, and with others present, just to name a few. For example, in one aspect, a valid state as determined by the ANN may be sitting with others present, or in-bed with caregiver present, or standing.

In one aspect, the input to the ANN is processed by the ANN. The output from the ANN is provided to a state machine application which analyzes the ANN output to determine the current state of the subject based on the ANN output, ANN output history, and previous state history. For example, a determined ANN output of “away” may update the state of the person and trigger an alert to family members or staff at a facility where the subject is located. Alternatively, a determined state of “fallen” may trigger an alert to a set of emergency contacts, such as a caregiver or family member. The determined state can also be saved into a memory and subsequently used to contribute towards a set of collected activity data on a person. Advantageously, an alert may be in the form of an audible or visual notification, a digital notification, a pre-recorded phone call, a text message, a computer sound, or other physical or digital notification or communication to alert one or more desired individuals of the subject's current state and/or location and/or position. Additionally, the type of alert may convey whether assistance is needed by the subject.

In one aspect, the ANN is continuously trained by continuous monitoring of the image data collected by the camera sensor. Each room may have a separately trained ANN instance so as to tune the ANN instance specifically to the unique environment. Training the ANN may advantageously improve the operation of all cameras in all rooms, but more importantly such training specifically improves the operation of a particular camera in a particular room. Accordingly, captured images from a particular room are continuously analyzed by human data labelers and/or algorithmic processes to confirm the various states of a subject within the room. This process for continuously tuning the ANN may include applying machine learning methods, such as neural network back propagation, to increase the accuracy of the learning model with the specifically collected dataset.

In one aspect, the complete system, including the camera, the ANN and the audible and visual notification hardware, is located in the room that is being monitored. In an alternative aspect, certain portions of the system may be located elsewhere, such as audible and visual notification hardware, and the processors that execute the ANN application and the state machine application.

In one aspect, the system includes a mobile application that allows a live feed or delayed feed of the field of view of the camera and current state detected by the state machine application. The mobile application may also allow views of still images captured by the camera sensor. Advantageously, the mobile application may also allow a user to configure different types of alerts based on customized criteria established by the user. The mobile application may also be configured to access a plurality of cameras in a variety of different rooms of a facility or different rooms at different facilities. The mobile application may also be configured to provide information about one or more subjects being monitored, for example, the amount of time a subject spent in-bed, the number of times a subject stands, the number of times and/or times of day a subject gets out of bed, and other helpful information as determined by the ANN as a result of the ongoing image analysis.

Other features and advantages of the present invention will become more readily apparent to those of ordinary skill in the art after reviewing the following detailed description and accompanying drawings.

### BRIEF DESCRIPTION OF THE DRAWINGS

The structure and operation of the present invention will be understood from a review of the following detailed description and the accompanying drawings in which like reference numerals refer to like parts and in which:

FIG. 1 illustrates an example infrastructure, in which one or more of the processes described herein may be implemented, according to an embodiment;

FIG. 2 illustrates an example processing system, by which one or more of the processes described herein may be executed, according to an embodiment;

FIG. 3 illustrates an example training process for an example artificial neural network, by which one or more of the processes described herein may be executed, according to an embodiment;

FIG. 4 illustrates an example operation of an example artificial neural network, by which one or more of the processes described herein may be executed, according to an embodiment;

FIG. 5 is a flow diagram illustrating an example process for determining a state of a subject according to an embodiment of the invention;

FIG. 6 is a flow diagram illustrating an example process for providing a state of a subject to a remote use according to an embodiment of the invention; and

FIG. 7 is a flow diagram illustrating an example process for continuous improvement of an artificial neural network according to an embodiment of the invention.

### DETAILED DESCRIPTION

Disclosed herein are systems, methods, and non-transitory computer-readable media for detecting changes in a position of a subject. For example, one method disclosed herein allows for one or more images of a room to be captured by a camera sensor and fed into an application including an ANN portion and a state machine portion for processing. The ANN portion of the application combined with the state machine portion of the application processes the image(s) to determine a current state of the subject. If the determined current state of the subject is associated with one or more alerts, the system delivers one or more alerts, which may be audible, visual, and/or haptic.

After reading this description it will become apparent to one skilled in the art how to implement the invention in various alternative embodiments and alternative applications. However, although various embodiments of the present invention will be described herein, it is understood that these embodiments are presented by way of example only, and not limitation. As such, this detailed description of various alternative embodiments should not be construed to limit the scope or breadth of the present invention as set forth in the appended claims.

### 1. System Overview

### 1.1. Infrastructure

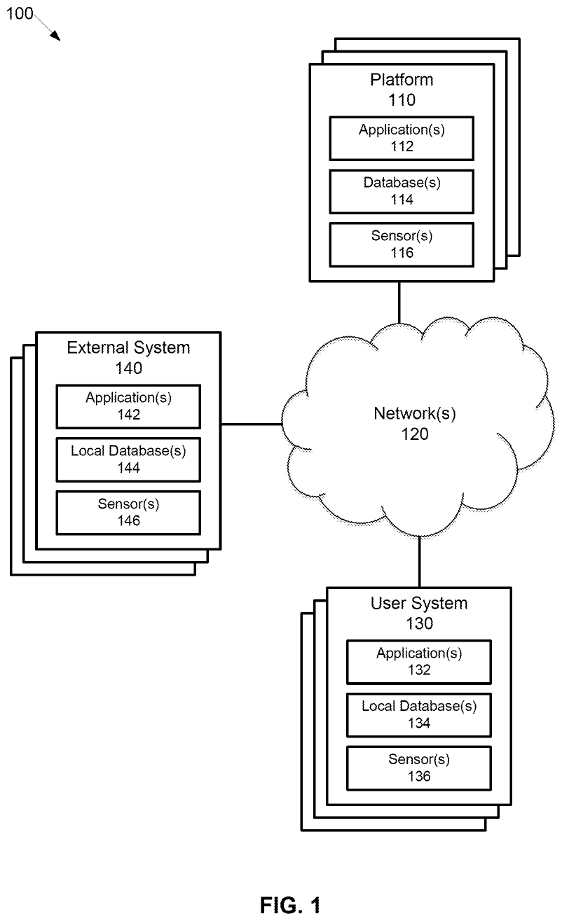

FIG. 1 illustrates an example infrastructure in which one or more of the disclosed processes may be implemented, according to an embodiment. The infrastructure may comprise a platform 110 (e.g., one or more servers) which hosts and/or executes one or more of the various functions, processes, methods, and/or software modules described herein. Platform 110 may comprise dedicated servers, or may instead comprise cloud instances, which utilize shared resources of one or more servers. These servers or cloud instances may be collocated and/or geographically distributed. Platform 110 may also comprise or be communicatively connected to a server application 112 and/or one or more databases 114 and/or one or more sensors 116. In addition, platform 110 may be communicatively connected to one or more user systems 130 (e.g., mobile devices, laptops, personal computers, etc.) via one or more networks 120. Platform 110 may also be communicatively connected to one or more external systems 140 (e.g., other platforms, websites, camera systems, etc.) via one or more networks 120.

Network(s) 120 may comprise the Internet, and platform 110 may communicate with user system(s) 130 through the Internet using standard transmission protocols, such as HyperText Transfer Protocol (HTTP), HTTP Secure (HTTPS), File Transfer Protocol (FTP), FTP Secure (FTPS), Secure Shell FTP (SFTP), and the like, as well as proprietary protocols. While platform 110 is illustrated as being connected to various systems through a single set of network(s) 120, it should be understood that platform 110 may be connected to the various systems via different sets of one or more networks. For example, platform 110 may be connected to a subset of user systems 130 and/or external systems 140 via the Internet, but may be connected to one or more other user systems 130 and/or external systems 140 via an intranet. Furthermore, while only a few user systems 130 and external systems 140, one server application 112, and one set of database(s) 114 are illustrated, it should be understood that the infrastructure may comprise any number of user systems, external systems, server applications, and databases.

User system(s) 130 may comprise any type or types of computing devices capable of wired and/or wireless communication, including without limitation, desktop computers, laptop computers, tablet computers, smart phones or other mobile phones, servers, game consoles, head mounted displays, televisions, set-top boxes, electronic kiosks, point-of-sale terminals, Automated Teller Machines, and/or the like. User system(s) 130 may include one or more application 132, databases 134 (e.g., a memory 134) and one or more sensors 136.

External system 140 may comprise any type of imaging system such as a camera system. External system 140 may be located in a room occupied by a subject. In one aspect, external system 140 includes one or more sensors 146 that are configured to capture image data. For example, the sensor 146 may be trained on a high traffic portion of the room that is occupied by the subject such as the bed and/or other furniture. The image data that is captured may be black and white image data, color image data, infra-red image data, and the like. The external system 140 is configured to communicate with the platform 110 via the network 120 and is further configured to transmit the information captured by the sensor, along with related meta data, over the network 120 to platform 110. In one aspect, the transmission of the image data and related meta data may occur via an application 142 that executes on the external system 140. For example, application 142 may be a firmware application written to directly transmit image data and meta data to platform 110 via the network 120. Additionally, external system 140 may capture and send image data and related meta data one image at a time or external system 140 may capture multiple images and related meta data and store the information in memory 144 and perform some level of computational preprocessing (e.g., resizing, down sampling, encryption, etc.) before transmitting the image data and related meta data to the platform 110.

In one aspect, platform 110 and external system 140 may be integrated into a single device. Alternatively, platform 110 may be deployed in the cloud while external system 140 is deployed in the room occupied by the subject.

In one aspect, the application 112 of the platform 110 is configured to process the information (e.g., image data and related meta data) received from the external system 140 to determine a state of the subject who occupies the room in which the external system 140 is deployed. The application 112 is further configured to store one or more states of the subject that are determined over time and, in certain circumstances when predetermined criteria a met, the application 112 is further configured to transmit an alert to one or more recipients. For example, an alert may be an indication that the subject has fallen. Alternatively, an alert may be an indication that the subject is awake.

In one aspect, application 112 is implemented as an artificial neural network (ANN) and includes state machine logic that is used to determine the state of the subject in one or more images.

In one aspect, an image that is captured by external system 140 comprises three dimensions, namely a height dimension, a width dimension, and color channels. For example, the three dimensions of an image may be represented as 224×224×3. Advantageously, the application 112 and/or application 142 is configured to apply certain image preprocessing techniques to the one or more images captured by the sensor of the external system 140 to modify, if necessary, the dimensions of the image in order to comply with the expected dimensions of an image that is input to the application 112 when implemented as an ANN. Such image preprocessing techniques may include resizing, center cropping, and channel collapsing.

In one aspect, the application 112 when implemented as an ANN may accept as input one or more images and process the one or more images through one or more of a convolutional neural network, recurrent neural network, and/or attention network for the purpose of classifying one or more subjects in the one or more images into one of five states. The application 112 when implemented as an ANN is trained to determine a confidence of the image being classified as one of five states and provide a confidence output, between 0 and 1, for each state. In one aspect, this may be accomplished using, e.g., a softmax ANN output layer.

In one aspect, the five states include: sitting, standing, in-bed, fall, or empty room. The names of the five states may be used as labels to describe the action of the subject in the image. In one aspect, an output from the ANN is provided for each of the five states and a confidence score for each state is also provided. Accordingly, the state machine portion of the application 112 receives as input a confidence score for the subject being in each of the five states.

In one aspect, captured images from external system 140 may be stored in memory 114 by application 112 and later be provided to a user system 130 upon request. In response to such a request by a user system 130, the application 112 is configured to transmit via network 120 one or more images stored in memory 114 to the requesting user system 130. Such transmitted images may be static images or a series of images may be transmitted to a requesting user system 130 as a live feed of the room that is occupied by the subject.

The state machine portion of the application 112 receives the current state of the subject from the ANN portion of the application 112 and stores the current state in memory 114. The current state of the subject is subsequently used by the application 112 to determine whether the subject has changed state. Advantageously, determining whether the subject has changed state allows the application 112 to store a time (or at least an approximate time) that a state change occurred. Additionally, a change in state may prompt the application 112 to trigger one or more alerts to one or more individuals such as a caregiver and/or a family member.

In one aspect, the state machine portion of application 112 may use the most confident output from the ANN (e.g., the highest confidence score) to determine which state the subject is in. Additionally, the state machine portion of application 112 is configured to initially determine whether the output of the ANN portion of application 112 is sufficiently confident to be considered. In one aspect, the state machine portion of application 112 applies a threshold to the confidence score such that, for example, if the highest confidence score is under 0.4, the confidence scores from the ANN portion of application 112 are not considered. In such a circumstance, the state machine portion of application 112 determines that the state of the subject remains unchanged. However, in such a circumstance, the application 112 may save the image received from external system 140 in memory 114 and subsequently be used for future ANN model training.

In one aspect, if the highest confidence score from the ANN portion of application 112 exceeds the threshold, the state machine portion of application 112 obtains previous outputs of the ANN portion of application 112 that have been stored in memory 114 and analyzes the previous outputs to determine whether a sufficient amount of time has elapsed to allow for a state change. The elapsed time can be measured literally or as a function of the number of ANN outputs that are analyzed. For example, if the external system 140 is capturing images at a rate of one per minute and the state machine portion of application 112 analyzes five prior outputs from the ANN portion of application 112 (e.g., five images were captured since the most recent state change), the literal passing of five minutes may be considered sufficient for the subject to have changed state. Alternatively, if the external system is capturing live feed video at a frame rate of 30 frames per second and the state machine portion of application 112 analyzes three hundred prior outputs from the ANN portion of application 112, the three hundred prior outputs may not be considered sufficient for the subject to have changed state.

Advantageously, the analysis performed by the state machine portion of application 112 functions as a small time buffer and applies a short delay before the application 112 changes the state of the subject. This allows the application 112 to determine that when the subject is in a new state compared to the current state for a sufficient period of time, the state of the subject is determined to have changed and the current state of the subject is updated in memory 114 to reflect the newly determined state of the subject.

In one aspect, when a change in the state of the subject has been determined by the state machine portion of application 112, the time of the state change and the current state are stored in memory 114. This allows the current state to always be available for sending to one or more user systems 130 upon request. Additionally, upon any state change, the current state is evaluated to identify any predetermine alerts that may correspond to the current state. Such alerts may then be sent by the application 112 via the network 120. Such alerts may be sent, e.g., to one or more user systems 130 and one or more external systems 140. For example, sending an alert to a user system 130 may notifying a subscribed user that the subject in the room has changed states.

In one aspect, time period may be allowed to elapse after a state of the subject has changed and before any alert is sent. This time period advantageously ensures that the subject has been in the new current state for a defined duration, such as one minute, before triggering an alert. The time period that is allowed to elapse may be unique to each state. For example, a fall alert may be sent immediately while a standing alert may be delayed for one minute before sending to determine if the standing state was a transitional state between, e.g., an in-bed state and a sitting state. In one aspect, the state machine portion of application 112 maintains a timer that tracks how long the subject is out of bed, meaning when they are in the standing, sitting, empty room, or fall state. Advantageously, this timer allows the application 112 to alert one or more individuals when the subject has been out of bed for a predetermined duration of time. Such an alert can facilitate assistance to a subject when the subject has not safely returned to bed in a normal period of time.

In one aspect, the application 112 may send one or more alerts to one or more user systems 130. Such alerts may be in the form of text messages, push notifications, application pop ups, and pre-recorded phone calls. In one aspect, the alert may trigger the user system 130 to deliver haptic feedback to a user of the user system 130. Advantageously, certain types of alerts, for example, alerts corresponding to the fall state and/or corresponding to the subject being out of bed for too long may trigger a pre-recorded phone call to the user system 130. Other alerts triggered by other state changes and/or other timers may trigger less intrusive push notifications and text messages.

In one aspect, the user system 130 is configured to receive alerts and notify the user of the user system 130. This may be accomplished via the application 132. Additionally, the application 132 may be configured to communicate with the application 112 on the platform 110 to obtain the current state of a subject and present the current state of the subject on a user interface of the user system 130. The application 132 of the user system 130 may also cooperate with the application 112 of the platform 110 to provide a live feed of the room where the subject is located such that images captured from the external system 140 in the room where the subject is located can be presented on a user interface of the user system 130. The application 132 may also cooperate with the application 112 to obtain and provide to a user of user system 130 certain historical state information about the subject and to select and configure certain alerts to be sent to the user system 130. For example, the application 132 may cooperate with the application 112 to allow a user of the user system 130 to configure one or more alerts to be sent to the user system 130 for certain state changes, certain elapsed timers, and to provide information about historical alerts, and to view certain analytics about the subject based on historical data such as image data and meta data and state data corresponding to the subject.

In one aspect, the application 132 allows the user of the user system 130 to opt into certain alerts such as state changes to sitting, standing, in-bed, fall, empty room, and elapsed timers for when the subject has been out of bed for an amount of time that exceeds a predetermined threshold, which may be customized for the particular subject.

In one aspect, if an external system 140 fails to communicate with the platform 110 for a certain period of time, an elapsed timer corresponding to that certain period of time may trigger an alert. Such an alert may advantageously include analytics of any image data and or meta data received from the external system 140 prior to the gap in communication.

In one aspect, the application 132 allows a user of the user system 130 to configure a blackout period during which alerts are not presented to the user of the user system 130, notwithstanding the fact that an alert was triggered and sent by the platform 110 to the user system 130. For example, the user may configure an alerting blackout period from 9am to 7pm. Advantageously, certain types of alerts, such as a fall alert, can be configured for immediate delivery, even during a blackout period that has been set by a user of the user system 130.

In one aspect, each external system 140 may be associated with one or more user accounts that are designated as an administrator for the external system 140. The administrator account allows an appropriate user to determine what other users and or users systems 130 may access information about the subject via platform 110.

In one aspect, multiple users who are not an administrator may be given access to view information about a subject. These additional users may be set up as caregiver users and associated with the external system 140 and allowed access to the data and information provided to the platform 110 by the external system 140.

In one aspect, the administrative user has additional privileges corresponding to a particular subject with respect to external system 140 that is in the room occupied by the particular subject. For example, the administrative user may be allowed to blur or disable the live feed from external system 140 or may be allowed to limit access to the live feed from external system 140 to certain users such as caregiver users or other users. This may advantageously preserve the privacy of the subject.

In one aspect, the application 132 on the user system 130 cooperates with the application 112 on the platform 110 to provide analytics about a subject that are based on the subject's state history. These analytics may include durations that a subject spent in each state during a certain time window and may include the number of times the subject was in each state during a certain time window. For example, such analytics may include the amount of time the subject spent in-bed and the number of times the subject transitioned from a different state to the in-bed state.

In one aspect, certain metrics about a subject may be presented by the application 132 on a user interface of the user system 130 as historical trend data and may also include comparative analytics that compare the subject against a larger population of subjects who are monitored by the platform 110 via separate external systems 140. In this fashion, caregivers and users of user systems 130 and even subjects can compare a particular subject against a larger population of subjects.

In one aspect, a safety/wellness score for an individual subject is generated by the application 112. For example, the safety/wellness score can be a numerical score between 0 and 100 that is a measure of how much time the subject spent in-bed. The score may be weighted such that riskier behaviors such as leaving the room have a more negative impact on the score versus less risky behaviors such as standing, which in turn has a larger negative impact on the score than sitting.

In one aspect, a user of a user system 130 can view via the application 132 a comparison of the amount of time a subject has spent in-bed, sitting, and out of bed. The application 132 is also configured to provide to a user of the user system 130 those certain hours within a twenty four hour period that have the highest frequency for the subject to be out of bed. Additionally, the application 132 is configured to provide a user of the user system 130 the number of falls that have been detected for a subject and the number of times the subject has been out of bed during a selected time period.

In one aspect, the application 132 is configured to provide the user of the user system 130 a daily update that summarizes the state changes and corresponding analytics from the previous night (or some other predetermined time period). The daily update may, for example, include information such as the safety/wellness score, the during of time the subject spent in-bed, the number of times the subject left the bed, and other desirable information that can be customized by the user of the user system 130.

Platform 110 may comprise web servers which host one or more websites and/or web services. In embodiments in which a website is provided, the website may comprise a graphical user interface, including, for example, one or more screens (e.g., webpages) generated in HyperText Markup Language (HTML) or other language. Platform 110 transmits or serves one or more screens of the graphical user interface in response to requests from user system(s) 130. In some embodiments, these screens may be served in the form of a wizard, in which case two or more screens may be served in a sequential manner, and one or more of the sequential screens may depend on an interaction of the user or user system 130 with one or more preceding screens. The requests to platform 110 and the responses from platform 110, including the screens of the graphical user interface, may both be communicated through network(s) 120, which may include the Internet, using standard communication protocols (e.g., HTTP, HTTPS, etc.). These screens (e.g., webpages) may comprise a combination of content and elements, such as text, images, videos, animations, references (e.g., hyperlinks), frames, inputs (e.g., textboxes, text areas, checkboxes, radio buttons, drop-down menus, buttons, forms, etc.), scripts (e.g., JavaScript), and the like, including elements comprising or derived from data stored in one or more databases (e.g., database(s) 114) that are locally and/or remotely accessible to platform 110. Platform 110 may also respond to other requests from user system(s) 130.

Platform 110 may further comprise, be communicatively coupled with, or otherwise have access to one or more database(s) 114. For example, platform 110 may comprise one or more database servers which manage one or more databases 114. A user system 130 or server application 112 executing on platform 110 may submit data (e.g., user data, form data, etc.) to be stored in database(s) 114, and/or request access to data stored in database(s) 114. Any suitable database may be utilized, including without limitation MySQL™, Oracle™, IBM™, Microsoft SQL™ Access™, PostgreSQL™, and the like, including cloud-based databases and proprietary databases. Data may be sent to platform 110, for instance, using the well-known POST request supported by HTTP, via FTP, and/or the like. This data, as well as other requests, may be handled, for example, by server-side web technology, such as a servlet or other software module (e.g., comprised in server application 112), executed by platform 110.

In embodiments in which a web service is provided, platform 110 may receive requests from user system(s) 130 and/or external system(s) 140, and provide responses in extensible Markup Language (XML), JavaScript Object Notation (JSON), and/or any other suitable or desired format. In such embodiments, platform 110 may provide an application programming interface (API) which defines the manner in which user system(s) 130 and/or external system(s) 140 may interact with the web service. Thus, user system(s) 130 and/or external system(s) 140 (which may themselves be servers), can define their own user interfaces, and rely on the web service to implement or otherwise provide the backend processes, methods, functionality, storage, and/or the like, described herein. For example, in such an embodiment, a client application 132 executing on one or more user system(s) 130 may interact with a server application 112 executing on platform 110 to execute one or more or a portion of one or more of the various functions, processes, methods, and/or software modules described herein. Client application 132 may be “thin,” in which case processing is primarily carried out server-side by server application 112 on platform 110. A basic example of a thin client application 132 is a browser application, which simply requests, receives, and renders webpages at user system(s) 130, while server application 112 on platform 110 is responsible for generating the webpages and managing database functions. Alternatively, the client application may be “thick,” in which case processing is primarily carried out client-side by user system(s) 130. It should be understood that client application 132 may perform an amount of processing, relative to server application 112 on platform 110, at any point along this spectrum between “thin” and “thick,” depending on the design goals of the particular implementation. In any case, the application described herein, which may wholly reside on either platform 110 (e.g., in which case server application 112 performs all processing) or user system(s) 130 (e.g., in which case client application 132 performs all processing) or be distributed between platform 110 and user system(s) 130 (e.g., in which case server application 112 and client application 132 both perform processing), can comprise one or more executable software modules that implement one or more of the processes, methods, or functions of the application described herein.

### 1.2. Example Processing Device

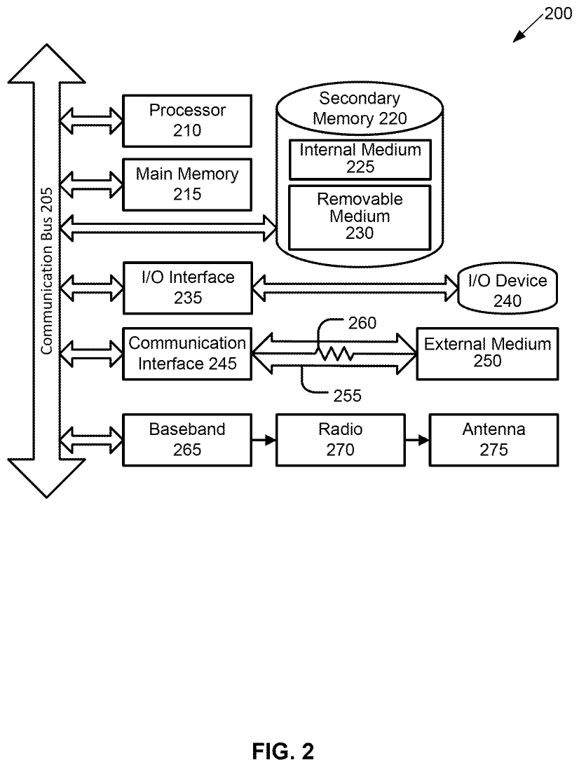

FIG. 2 is a block diagram illustrating an example wired or wireless system 200 that may be used in connection with various embodiments described herein. For example, system 200 may be used as or in conjunction with one or more of the functions, processes, or methods (e.g., to store and/or execute the application or one or more software modules of the application) described herein, and may represent components of platform 110, user system(s) 130, external system(s) 140, and/or other processing devices described herein. System 200 can be a server or any conventional personal computer, or any other processor-enabled device that is capable of wired or wireless data communication. In one aspect, system 200 can be a camera system having the field of view of an imaging sensor trained on at least a portion of a room. Other computer systems and/or architectures may be also used, as will be clear to those skilled in the art.

System 200 preferably includes one or more processors, such as processor 210. Additional processors may be provided, such as an auxiliary processor to manage input/output, an auxiliary processor to perform floating-point mathematical operations, a special-purpose microprocessor having an architecture suitable for fast execution of signal-processing algorithms (e.g., digital-signal processor), a slave processor subordinate to the main processing system (e.g., back-end processor), an additional microprocessor or controller for dual or multiple processor systems, and/or a coprocessor. Such auxiliary processors may be discrete processors or may be integrated with processor 210. Examples of processors which may be used with system 200 include, without limitation, the Pentium® processor, Core i7® processor, and Xeon® processor, all of which are available from Intel Corporation of Santa Clara, California.

Processor 210 is preferably connected to a communication bus 205. Communication bus 205 may include a data channel for facilitating information transfer between storage and other peripheral components of system 200. Furthermore, communication bus 205 may provide a set of signals used for communication with processor 210, including a data bus, address bus, and/or control bus (not shown). Communication bus 205 may comprise any standard or non-standard bus architecture such as, for example, bus architectures compliant with industry standard architecture (ISA), extended industry standard architecture (EISA), Micro Channel Architecture (MCA), peripheral component interconnect (PCI) local bus, standards promulgated by the Institute of Electrical and Electronics Engineers (IEEE) including IEEE 488 general-purpose interface bus (GPIB), IEEE 696/S-100, and/or the like.

System 200 preferably includes a main memory 215 and may also include a secondary memory 220. Main memory 215 provides storage of instructions and data for programs executing on processor 210, such as one or more of the functions and/or modules discussed herein. It should be understood that programs stored in the memory and executed by processor 210 may be written and/or compiled according to any suitable language, including without limitation C/C++, Java, JavaScript, Perl, Visual Basic, .NET, and the like. Main memory 215 is typically semiconductor-based memory such as dynamic random access memory (DRAM) and/or static random access memory (SRAM). Other semiconductor-based memory types include, for example, synchronous dynamic random access memory (SDRAM), Rambus dynamic random access memory (RDRAM), ferroelectric random access memory (FRAM), and the like, including read only memory (ROM).

Secondary memory 220 may optionally include an internal medium 225 and/or a removable medium 230. Removable medium 230 is read from and/or written to in any well-known manner. Removable storage medium 230 may be, for example, a magnetic tape drive, a compact disc (CD) drive, a digital versatile disc (DVD) drive, other optical drive, a flash memory drive, and/or the like.

Secondary memory 220 is a non-transitory computer-readable medium having computer-executable code (e.g., disclosed software modules) and/or other data stored thereon. The computer software or data stored on secondary memory 220 is read into main memory 215 for execution by processor 210.

In alternative embodiments, secondary memory 220 may include other similar means for allowing computer programs or other data or instructions to be loaded into system 200. Such means may include, for example, a communication interface 245, which allows software and data to be transferred from external storage medium 250 to system 200. Examples of external storage medium 250 may include an external hard disk drive, an external optical drive, an external magneto-optical drive, and/or the like. Other examples of secondary memory 220 may include semiconductor-based memory, such as programmable read-only memory (PROM), erasable programmable read-only memory (EPROM), electrically erasable read-only memory (EEPROM), and flash memory (block-oriented memory similar to EEPROM).

As mentioned above, system 200 may include a communication interface 245. Communication interface 245 allows software and data to be transferred between system 200 and external devices (e.g. printers), networks, or other information sources or information recipients. For example, computer software or executable code may be transferred to system 200 from a network server (e.g., platform 110) via communication interface 245 and digital information such as digital image files and digital video content may be transferred from system 200 to a network server (e.g., platform 110) via communication interface 245. Examples of communication interface 245 include a built-in network adapter, network interface card (NIC), Personal Computer Memory Card International Association (PCMCIA) network card, card bus network adapter, wireless network adapter, Universal Serial Bus (USB) network adapter, modem, a wireless data card, a communications port, an infrared interface, an IEEE 1394 fire-wire, and any other device capable of interfacing system 200 with a network (e.g., network(s) 120) or another computing device. Communication interface 245 preferably implements industry-promulgated protocol standards, such as Ethernet IEEE 802 standards, Fiber Channel, digital subscriber line (DSL), asynchronous digital subscriber line (ADSL), frame relay, asynchronous transfer mode (ATM), integrated digital services network (ISDN), personal communications services (PCS), transmission control protocol/Internet protocol (TCP/IP), serial line Internet protocol/point to point protocol (SLIP/PPP), and so on, but may also implement customized or non-standard interface protocols as well.

Software and data transferred via communication interface 245 are generally in the form of electrical communication signals 260. These signals 260 may be provided to communication interface 245 via a communication channel 255. In an embodiment, communication channel 255 may be a wired or wireless network (e.g., network(s) 120), or any variety of other communication links. Communication channel 255 carries signals 260 and can be implemented using a variety of wired or wireless communication means including wire or cable, fiber optics, conventional phone line, cellular phone link, wireless data communication link, radio frequency (“RF”) link, or infrared link, just to name a few.

Computer-executable code (e.g., computer programs, such as the disclosed application, or software modules) is stored in main memory 215 and/or secondary memory 220. Computer programs can also be received via communication interface 245 and stored in main memory 215 and/or secondary memory 220. Such computer programs, when executed, enable system 200 to perform the various functions of the disclosed embodiments as described elsewhere herein.

In this description, the term “computer-readable medium” is used to refer to any non-transitory computer-readable storage media used to provide computer-executable code and/or other data to or within system 200. Examples of such media include main memory 215, secondary memory 220 (including internal memory 225, removable medium 230, and external storage medium 250), and any peripheral device communicatively coupled with communication interface 245 (including a network information server or other network device). These non-transitory computer-readable media are means for providing executable code, programming instructions, software, and/or other data to system 200.

In an embodiment that is implemented using software, the software may be stored on a computer-readable medium and loaded into system 200 by way of removable medium 230, I/O interface 235, or communication interface 245. In such an embodiment, the software is loaded into system 200 in the form of electrical communication signals 260. The software, when executed by processor 210, preferably causes processor 210 to perform one or more of the processes and functions described elsewhere herein.

In an embodiment, I/O interface 235 provides an interface between one or more components of system 200 and one or more input and/or output devices 240. Example input devices include, without limitation, sensors, such as a camera or other imaging sensor, keyboards, touch screens or other touch-sensitive devices, biometric sensing devices, computer mice, trackballs, pen-based pointing devices, and/or the like. Examples of output devices include, without limitation, other processing devices, cathode ray tubes (CRTs), plasma displays, light-emitting diode (LED) displays, liquid crystal displays (LCDs), printers, vacuum fluorescent displays (VFDs), surface-conduction electron-emitter displays (SEDs), field emission displays (FEDs), head mounted displays (HMDs), and/or the like. In some cases, an input and output device 240 may be combined, such as in the case of a touch panel display (e.g., in a smartphone, tablet, or other mobile device).

In an embodiment, the I/O device 240 may be any type of external or integrated display and may include one or more discrete displays that in aggregate form the I/O device 240. The I/O device 240 may be capable of 2D or 3D presentation of visual information to a user of the system 200. In one embodiment, the I/O device 240 may be a virtual reality or augmented reality device in the form of HMD by the user so the user may visualize the presentation of information in 3D.

System 200 may also include optional wireless communication components that facilitate wireless communication over a voice network and/or a data network (e.g., in the case of user system 130). The wireless communication components comprise an antenna system 275, a radio system 270, and a baseband system 265. In system 200, radio frequency (RF) signals are transmitted and received over the air by antenna system 275 under the management of radio system 270.

In an embodiment, antenna system 275 may comprise one or more antennae and one or more multiplexors (not shown) that perform a switching function to provide antenna system 275 with transmit and receive signal paths. In the receive path, received RF signals can be coupled from a multiplexor to a low noise amplifier (not shown) that amplifies the received RF signal and sends the amplified signal to radio system 270.

In an alternative embodiment, radio system 270 may comprise one or more radios that are configured to communicate over various frequencies. In an embodiment, radio system 270 may combine a demodulator (not shown) and modulator (not shown) in one integrated circuit (IC). The demodulator and modulator can also be separate components. In the incoming path, the demodulator strips away the RF carrier signal leaving a baseband receive audio signal, which is sent from radio system 270 to baseband system 265.

If the received signal contains audio information, then baseband system 265 decodes the signal and converts it to an analog signal. Then the signal is amplified and sent to a speaker. Baseband system 265 also receives analog audio signals from a microphone. These analog audio signals are converted to digital signals and encoded by baseband system 265. Baseband system 265 also encodes the digital signals for transmission and generates a baseband transmit audio signal that is routed to the modulator portion of radio system 270. The modulator mixes the baseband transmit audio signal with an RF carrier signal, generating an RF transmit signal that is routed to antenna system 275 and may pass through a power amplifier (not shown). The power amplifier amplifies the RF transmit signal and routes it to antenna system 275, where the signal is switched to the antenna port for transmission.

Baseband system 265 is also communicatively coupled with processor 210, which may be a central processing unit (CPU). Processor 210 has access to data storage areas 215 and 220. Processor 210 is preferably configured to execute instructions (i.e., computer programs, such as the disclosed application, or software modules) that can be stored in main memory 215 or secondary memory 220. Computer programs can also be received from baseband processor 260 and stored in main memory 210 or in secondary memory 220, or executed upon receipt. Such computer programs, when executed, enable system 200 to perform the various functions of the disclosed embodiments.

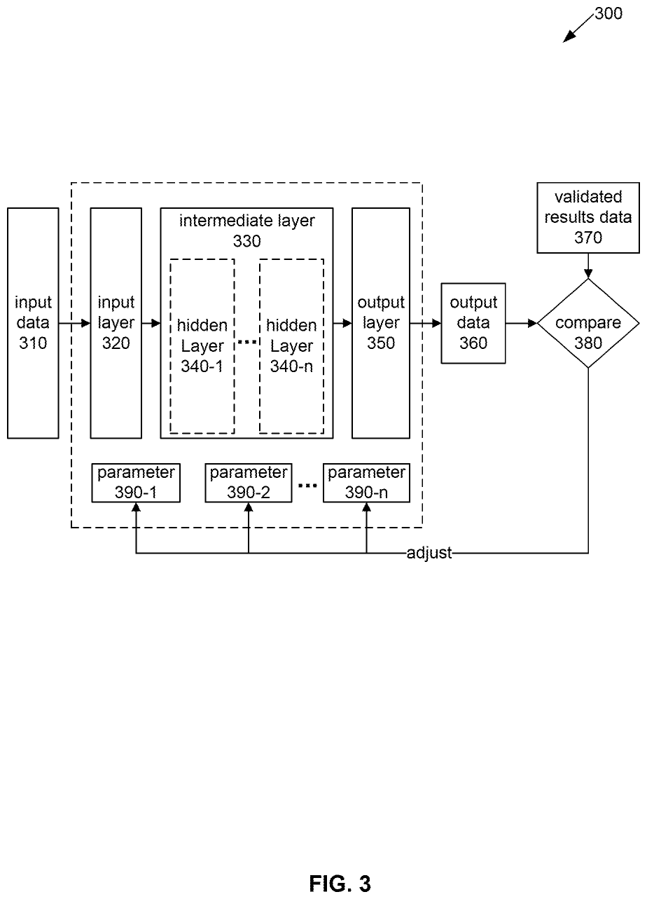

FIG. 3 illustrates an example training process for an example artificial neural network (ANN) 300, by which one or more of the processes described herein may be executed, according to an embodiment. In the illustrated embodiment, the ANN 300 receives input data 310 (e.g., an image captured by external system 140) at the input layer 320. The input layer 320 processes the input data to generate one or more outputs that are provided to the intermediate layer 330. As the input layer 320 processes the input data 310, the input layer 320 may use one or more parameters 390 (e.g., parameter 390-1, parameter 390-2, . . . , parameter 390-n) in the processing.

The intermediate layer 330 may comprise a plurality of hidden layers 340 (e.g., layer 340-1, . . . , layer 340-n). Each hidden layer 340 of the intermediate layer 330 receives one or more inputs from the input layer 320 or another hidden layer 340 and processes the one or more inputs to generate one or more outputs that are provided to another hidden layer 340 or to the output layer 350. As each hidden layer 340 performs its processing, the respective hidden layer 340 may use one or more parameters 390 (e.g., parameter 390-1, parameter 390-2, . . . , parameter 390-n) in the processing. The output layer 350 processes all of the inputs it receives from the various hidden layers 340 of the intermediate layer 330 and generates output data 360 comprising a confidence score associated with each of the five states that the subject in the one or more images processed by the ANN may be in. The output data 360 is compared to validated input data 370 (e.g., the known state of the subject in the input image data) and the results of the comparison 380 are used to adjust one or more parameters 390. Advantageously, the adjusted parameters 390 operate to improve the subsequent processing of input data 310 by the ANN 300 to generate more accurate output data 360.

In one aspect, the system 100 employs an Artificial Neural Network (ANN), for example embodied in application 112, to analyze sensor data. The sensor data is primarily one or more images captured by one or more cameras embedded in one or more external systems 140. The input to the ANN is typically a preprocessed image, structured to align with the ANN's requirements with respect to the dimensions of an image.

In one aspect, image preprocessing includes a series of operations applied to a captured image, which is intrinsically three-dimensional, having a height dimension, a width dimension, and color channels (e.g., 224×224×3). The series of operations may include image resizing, center cropping, and channel collapsing, and these operations are aimed at tailoring each image captured by an external system 140 to match the ANN's input specifications.

In one aspect, the ANN may be implemented using architectures such as a Convolutional Neural Network (CNN), a Recurrent Neural Network (RNN), or a Transformer Neural Network (TNN). The ANN is configured to process an image or a sequence of images to determine the state of a subject in the room where the external system 140 is located. The potential states into which the subject may be classified include sitting, standing, in-bed, fall, or empty room. The output of the ANN is a 1×5 dimensional vector the comprises the five states into which the subject may be classified and a confidence score (e.g., ranging from 0 to 1) associated with each state.

To improve accuracy of the ANN over time, the ANN undergoes an initial training phase using a large dataset of images, such as ImageNet with known states for each image. Subsequent to the initial training, the ANN is further trained by using images collected of a specific room during installation of the external system 140. In one aspect, this additional training comprises collecting approximately 500 to 1000 images per state of a sample subject in the particular room where the external system 140 is located.

Post-installation, when the system is deployed and operational, the system continuously collects image data from the particular room where the external system 140 is located and at least a portion of such images are processed to confirm the state corresponding to each image and the processed images and their known states are provided to the ANN for additional training. A portion of the software 112 on the platform 110 determines the conditions for subsequent data collection, for example when singular or average confidence levels determined by the ANN fall below a certain threshold.

Advantageously, in one aspect, human annotators may be employed to review the additional image data to confirm the state of an image to be used for additional training of the system. For example, human annotators may be used to review images with a confidence level below a certain threshold or images that are determined algorithmically or determine by a human annotator to be unique compared to existing images in the training set. Images that have been reviewed by a human annotator may be stored in memory 114 in association with their confirmed state until such time as a sufficiently large number of images and confirmed states has been accumulated. At that time, the ANN can be trained using the additional images and confirmed states. This iterative process of additional image collection, confirming of the corresponding state for each image, and additional training of the ANN may advantageously continue as long as there is more data to be collected and confirmed.

In one aspect, each installed external system 140 has a unique set of ANN parameters/weights, specifically trained on data gathered from its specific room. Alternatively, or in combination, a global ANN model may exist with all external systems 140 sharing the same ANN parameters/weights for the processing of images by the application 112. In this fashion, additional images that are collected and associated with confirmed states across all external systems 140 may be used for subsequent training of the global ANN model.

In one aspect, to account for the potential that conditions in a particular room may change over time, such as a change in the occupant and the presence of a new subject or a significant decrease in model confidence, a specialized ANN that was trained on the specific room can undergo additional training using new data, for example image data captured after the new occupant has arrived. The flexibility provided by the potential to retrain a specialized ANN previously trained on a specific room ensures the sustained accuracy of the ANN despite changes in the room or the subject who occupies the room.

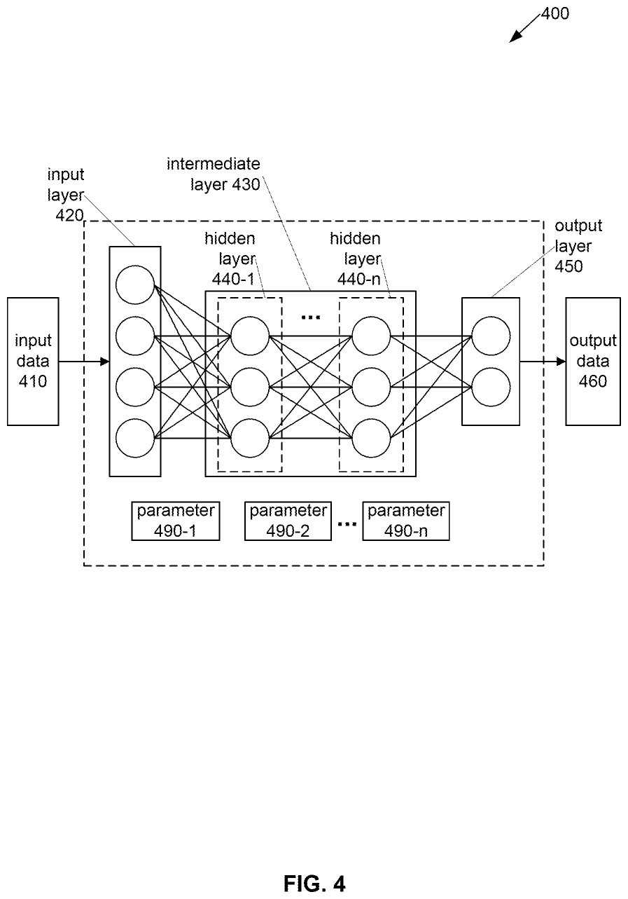

FIG. 4 illustrates an example operation of an example artificial neural network 400, by which one or more of the processes described herein may be executed, according to an embodiment. In the illustrated embodiment, the ANN 400 receives input data 410 (e.g., an image from external system 140) at the input layer 420. The input layer 420 processes the input data to generate one or more outputs that are provided to the intermediate layer 430. As the input layer 420 processes the input data 410, the input layer 420 may use one or more parameters 490 (e.g., parameter 490-1, parameter 490-2, . . . , parameter 490-n) in the processing.

The intermediate layer 430 may comprise a plurality of hidden layers 440 (layer 440-1, . . . , layer 440-n). Each hidden layer 440 of the intermediate layer 430 receives one or more inputs from the input layer 420 or another hidden layer 440 and processes the one or more inputs to generate one or more outputs that are provided to another hidden layer 440 or to the output layer 450. As each hidden layer 440 performs its processing, the respective hidden layer 440 may use one or more parameters 490 (parameter 490-1, parameter 490-2, . . . , parameter 490-n) in the processing. The output layer 450 processes all of the inputs it receives from the various hidden layers 440 of the intermediate layer 430 and generates output data 460 comprising a confidence score associated with each of the five states that the subject in the one or more images processed by the ANN may be in.

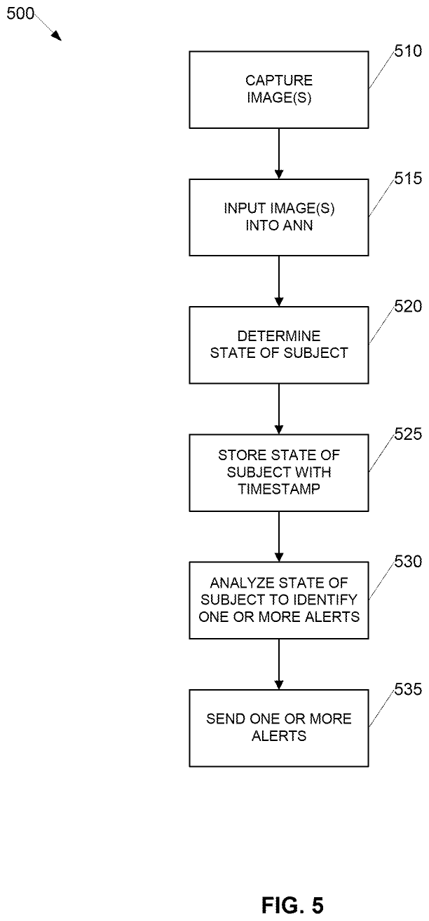

FIG. 5 is a flow diagram illustrating an example process 500 for determining a state of a subject according to an embodiment of the invention. In one aspect, the process of FIG. 5 may be carried out by the system described with respect to FIG. 1 in combination with one of more processing devices described with respect to FIG. 2 that may be executing artificial neural networks that are trained and operated as described in FIGS. 3 and 4.

Initially, at 510 the system captures one or more images. The image capture is done by a camera device that is positioned in a room. The imaging sensor of the camera device is advantageously trained on a desired portion of the room, for example, a portion that includes the bed and perhaps other furniture that may be commonly occupied by the subject who is the occupant of the room.

The images that are captured at 510 may be single still images or may be a series of images at a sufficiently high frame rate (e.g., 30 frames per second) to be considered digital video. The images may be color, black and white, infrared, or some other type of image that the sensor of the camera device is capable of capturing.

Next, at 515 the image (or images) are provided as an input to the artificial neural network. In one aspect, images are captured by the camera device and sent via a network to a server (e.g., platform 110) where the images are provided as input to the ANN (e.g., application 112).

Next, at 520, the ANN processes the images to determine a current state of the subject. In one aspect, there are 5 states that a subject may be in at any given time. Those states include: (1) in-bed; (2) sitting; (3) standing; (4) away; and (5) fall. For example, when an image or series of images includes a single individual and that individual is on the bed (covered by blankets or otherwise), the (1) in-bed state is determined. Alternatively, when an image or series of images includes a single individual and that individual is sitting on furniture or on the floor, the (2) sitting state is determined. Alternatively, when an image or series of images includes a single individual and that individual is standing in the room or walking around the room, the (3) standing state is determined. Alternatively, when an image or series of images does not include any individual, the (4) away state is determined. Finally, when an image or series of images includes a single individual and that individual has their torso or hand on the floor, the (5) fall state is determined.

In one aspect, an image or series of images may include more than one individual. When there is more than one individual in an image or series of images, if one individual has their torso or hand on the floor, the (5) fall state is determined, regardless of the other potential states that other individuals in the image(s) may be in. Alternatively, if no individual is in the (5) fall state but an individual is in-bed, the (1) in-bed state is determined, regardless of the other potential states that other individuals in the image(s) may be in. Alternatively, if there is no individual in the (5) fall state and there is no individual in-bed, but there is a person sitting, the (2) sitting state is determined, regardless of the other potential states that other individuals in the image(s) may be in. Alternatively, if all individuals in the image(s) are standing, the (3) standing state is determined.

Next, at 525, the current state of the subject as determined by the combination of the ANN portion of the application 112 and the state machine portion of the application 112 based on the analysis of the image or series of images, the system saves the determined state of the subject in association with a timestamp to generate a record of the state of the subject at a particular date and time.

Next, at 530, the system analyzes the determined current state of the subject to determine if there are one or more alerts associated with the determined current state of the subject. For example, a user of the system who is a family member of the subject, may create a custom alert to notify the user when the subject is standing or sitting so that the user is aware when the subject is likely awake and alert and ready for a visitor.

Finally, at 535, the system generates and sends one or more alerts that may be associated with the determined current state of the subject based on the analysis of the one or more images performed by the ANN.

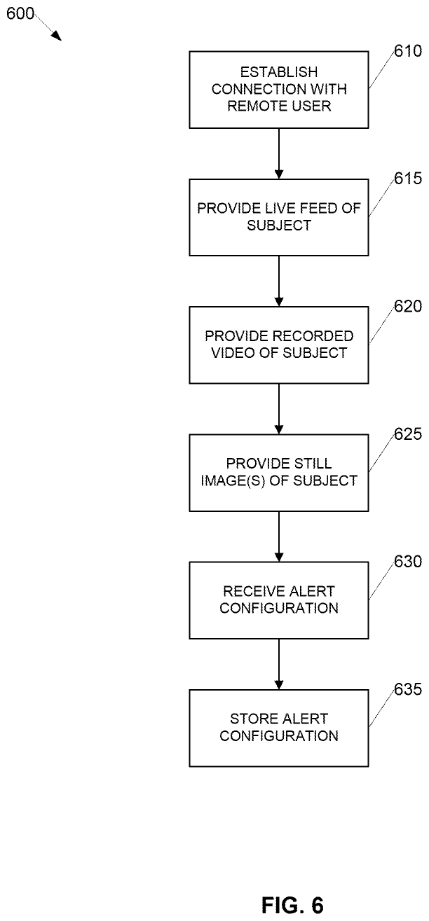

FIG. 6 is a flow diagram illustrating an example process 600 for providing a state of a subject to a remote use according to an embodiment of the invention. In one aspect, the process of FIG. 6 may be carried out by the system described with respect to FIG. 1 in combination with one of more processing devices described with respect to FIG. 2 that may be executing artificial neural networks that are trained and operated as described in FIGS. 3 and 4. Initially, at 610, the system establishes a connection with a remote user. The remote user may be using a user device 130 and the application 112 may establish a connection via a network 120 with the application 132 on the user device 130.

Next, at 615, the system optionally provides via the network 120 a live feed of the subject (or the subject's room), if such content was requested by the user system 130.

Next, at 620, the system optionally provides via the network 120 a recorded video of the subject (or the subject's room), if such content was requested by the user system 130.

Next, at 625, the system optionally provides via the network 120 a still image of the subject (or the subject's room), if such content was requested by the user system 130.

Next at 630, the system receives from the application 132 a configuration defining when an alert is requested. For example, the application 132 may submit criteria for when an alert is requested, such as when the subject has transitioned to a fall state or when the subject has transitioned from the in-bed state to a different state. An alert configuration may also be based on a timer or some other collected or analytic data, for example, if the subject has not been in the in-bed state for a predetermined length of time.

Next, at 635, in response to receiving the alert configuration, the system stores the alert configuration in association with the subject and the requesting user.

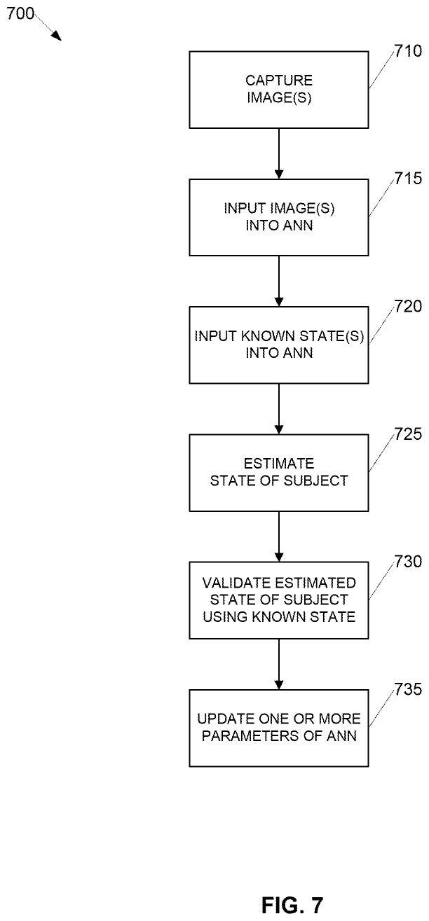

FIG. 7 is a flow diagram illustrating an example process 700 for continuous improvement of an artificial neural network according to an embodiment of the invention. In one aspect, the process of FIG. 7 may be carried out by the system described with respect to FIG. 1 in combination with one of more processing devices described with respect to FIG. 2 that may be executing artificial neural networks that are trained and operated as described in FIGS. 3 and 4.

Initially, at 710, the external system 140 captures one or more images and those images are validated to confirm the state of the subject in each of the one or more images. The validation may be done by a human annotator or by a separately trained ANN. Advantageously, the additional images captured by the external system 140 are specific to a particular room and therefore the images may be captured at different times of day to account for different ambient lighting conditions during daytime and nighttime hours.

Next, at 715, the one or more images are input into the ANN assigned to the particular room. Alternatively, in the case of a global ANN model, the one or more images are input into the global ANN.

Next, at 720, the known state (confirmed state) for each of the one or more images are also input into the ANN assigned to the particular room. Alternatively, in the case of a global ANN model, the known state (confirmed state) for each of the one or more images are input into the global ANN.

Next at 725, the ANN estimates the state for the one or more images that were input at 715.

Next, at 730 the estimated state for each of the one or more images as determined at 725 is validated against the known state for each of the one or more images to determine the accuracy of the ANN estimate. In certain circumstances, one or more parameters or weights used by the ANN in generating the estimates may be revised to improve the accuracy of the ANN when processing future images and generating estimates of the state of the subject in those future images.

Next, at 735, any revised parameters or weights are updated in the ANN to improve the accuracy and improve the confidence scores of future image processing by the ANN.

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

1. A system for detecting a position of a subject, comprising:
a camera system having a camera sensor trained on a portion of a room occupied by a subject;
an image preprocessing system configured to receive one or more images captured by the camera system and preprocess the one or more images to meet predetermined image characteristics;
an artificial neural network (ANN) system configured to receive the one or more preprocessed images and estimate a confidence score corresponding to each of five states of the subject in the one or more images, wherein five states of the subject include:
an empty room state;
an in-bed state;
a sitting state;
a standing state; and
a fall state;
a state machine system configured to receive the estimated confidence scores for each of the five states from the ANN and analyze the estimated confidence scores in combination with one or more previously determined states of the subject and a current state of the subject to determine a new current state of the subject; and
an alert system configured to receive the new current state of the subject and identify one or more alerts corresponding to the new current state of the subject and send the one or more alerts to one or more recipients via a data communication network.

2. The system of claim 1, wherein the one or more captured images comprise a series of still images captured at regular time intervals.

3. The system of claim 1, wherein the preprocessing system is further configured to:
crop a length of the one or more images;
crop a height of the one or more images; and
adjust a color of the one or more images.

4. The system of claim 1, wherein the alert system is further configured to send at least one of a digital notification, a visual notification, an audible notification, and a haptic notification.

5. The system of claim 1, wherein the alert system is further configured to send one of: a text message, an email, a push notification, and an application pop up.

6. The system of claim 1, wherein the alert system is further configured to send one of: a computer sound and a prerecorded phone call.

7. The system of claim 1, wherein the alert system is further configured to send a haptic vibration.

8. The system of claim 1, wherein a first of the one or more images includes two or more individuals, and wherein the state machine system is further configured to:
determine the new current state of the subject to be the fall state when one individual has their torso or hand on a floor;
if the fall state is not determined, determine the new current state of the subject to be the in-bed state when an individual is in-bed;
if one of the fall state and the in-bed state is not determined, determine the new current state of the subject to be the sitting state when an individual is sitting; and
if one of the fall state, the in-bed state, and the sitting state is not determined, determine the new current state of the subject to be the standing state when all individuals are standing.

9. The system of claim 1, wherein the alert system is further configured to alert one or more individuals when the state of the subject has not been in-bed for a predetermined duration of time.

10. The system of claim 1, wherein the ANN is further configured to receive a plurality of images captured of the room over a period of time and a validated state corresponding to each image and process the plurality of images and their corresponding validated states to retrain the ANN.

11. A method for detecting a position of a subject, comprising:
capturing one or more images by a camera system having a camera sensor trained on a portion of a room occupied by a subject;
preprocessing the one or more images captured by the camera system to meet predetermined image characteristics;
processing the one or more preprocessed images to estimate a confidence score corresponding to each of five states of the subject in the one or more images, wherein the five states of the subject include:
an empty room state;
an in-bed state;
a sitting state;
a standing state; and
a fall state;
analyzing the estimated confidence scores for each of the five states in combination with one or more previously determined states of the subject and a current state of the subject to determine a new current state of the subject;
identifying one or more alerts corresponding to the new current state of the subject; and
sending the one or more alerts to one or more recipients via a data communication network.

12. The method of claim 11, wherein the one or more captured images provide a video feed of the room occupied by the subject.

13. The method of claim 11, wherein the one or more captured images comprise a series of still images captured at regular time intervals.

14. The method of claim 11, wherein preprocessing further comprises:
cropping a length of the one or more images;
cropping a height of the one or more images; and
adjusting a color of the one or more images.

15. The method of claim 11, wherein sending one or more alerts to one or more recipients includes sending at least one of a digital notification, a visual notification, an audible notification, and a haptic notification.

16. The method of claim 15, wherein sending one or more digital notifications comprises sending one of: a text message, an email, a push notification, and an application pop up, wherein sending one or more audible notifications comprises sending one of:
a computer sound and a pre-recorded phone call, and wherein sending one or more haptic notifications comprises sending a vibration.

17.-18. (canceled)

19. The method of claim 11, wherein a first of the one or more images includes two or more individuals, further comprising:
determining the new current state of the subject to be the fall state when one individual has their torso or hand on a floor;
if the fall state is not determined, determining the new current state of the subject to be the in-bed state when an individual is in-bed;
if one of the fall state and the in-bed state is not determined, determining the new current state of the subject to be the sitting state when an individual is sitting; and
if one of the fall state, the in-bed state, and the sitting state is not determined, determining the new current state of the subject to be the standing state when all individuals are standing.

20. The method of claim 11, further comprising alerting one or more individuals when the state of the subject has not been in-bed for a predetermined duration of time.

21. The method of claim 11, further comprising receiving a plurality of images captured of the room over a period of time and a validated state corresponding to each image and processing the plurality of images and their corresponding validated states to retrain an artificial neural network configured to process the one or more preprocessed images to estimate confidence scores corresponding to a state of the subject.

22.-32. (canceled)

33. A non-transitory computer readable medium having stored thereon one or more sequences of instructions for causing one or more processors to perform steps for detecting a position of a subject, the steps comprising:
capturing one or more images by a camera system having a camera sensor trained on a portion of a room occupied by a subject;
preprocessing the one or more images captured by the camera system to meet predetermined image characteristics;
processing the one or more preprocessed images to estimate a confidence score corresponding to each of five states of the subject in the one or more images, wherein five states of the subject include:
an empty room state;
an in-bed state;
a sitting state;
a standing state; and
a fall state;
analyzing the estimated confidence scores for each of the five states in combination with one or more previously determined states of the subject and a current state of the subject to determine a new current state of the subject;
identifying one or more alerts corresponding to the new current state of the subject; and
sending the one or more alerts to one or more recipients via a data communication network.

34.-43. (canceled)
